(19) United States
(12) Patent Application Publication
(10) Pub. No.: US 2011/0182807 A1
(43) Pub. Date: Jul. 28, 2011

(54) Method and Dosage Form to Confirm Compliant Use of a Bioactive Agent

(76) Inventors: Richard C. Fuisz, Beverly Hills, CA (US); Joseph M. Fuisz, Surfside, FL (US)

(21) Appl. No.: 12/862,515

(22) Filed: Aug. 24, 2010

Related U.S. Application Data
(63) Continuation-in-part of application No. 12/704,742, filed on Feb. 12, 2010.
Application No. 12/704,742 is a continuation-in-part of application No. 12/692,737, filed on Jan. 25, 2010.

(51) Int. Cl. A61K 51/00; A61K 49/00; A61K 9/48; A61K 9/70; A61P 43/00

(52) U.S. Cl. 424/1.11; 424/9.2; 424/451; 424/444

(57) ABSTRACT

A dosage form includes a dose of at least one bioactive agent and a substance that can be readily confirmed by medical personnel to determine if the patient is compliant with taking the dosage form. The substance is at least one member selected from the group consisting of (a) a material incorporated in the dose in a manner that when the dosage form is properly administered is by itself or when combined with a reagent visible in the buccal, vaginal, nasal, rectal, aural, ophthalmic or ostomic cavity of a patient or an excretion of a patient in normal light or when exposed to a special frequency light source, (b) a material that is by itself not readily visible in the buccal, vaginal, nasal, rectal, aural, ophthalmic or ostomic cavity of a patient or an excretion of a patient in normal light but by itself or when combined with a reagent is readily visible when exposed to a special frequency light source, (c) a material that is by itself not readily visible in the buccal, vaginal, nasal, rectal, aural, ophthalmic or ostomic cavity of a patient or an excretion of a patient in normal light but when combined with a reagent is readily visible in normal light and (d) a material that is by itself not readily visible in the buccal, vaginal, nasal, rectal, aural, ophthalmic or ostomic cavity of a patient or an excretion of a patient in normal light but by itself or when combined with a reagent is readily detected by a non-visual detection method.

### CROSS-REFERENCE TO RELATED APPLICATION

This application is a continuation-in-part of prior application Ser. No. 12/704,742, filed Feb. 12, 2010, which is a continuation-in-part of prior application Ser. No. 12/692,737, filed Jan. 25, 2010, the contents of each of which are incorporated herein by reference.

### BACKGROUND OF THE INVENTION

Misuse of bioactive agents is a significant public health issue, particularly in the case of opiates and other drug classes that are subject to abuse. Broadly speaking, such abuse may take a number of forms. The patient may not use the prescribed drug at all and instead divert the drug for illicit resale. A patient or other user may misuse the drug in a manner other than its intended use, for example by hoarding the drug and then taking a large amount in a single administration to get “high.” Alternatively, the patient may take a drug intended for oral use and snort the drug or “mainline” the drug through injection. In other instances, an individual may uses basic chemical processes to separate various components of a drug dosage form so as to misuse a selected portion thereof.

Of course, non compliance with intended therapeutic regimes is not limited to “abuse” drugs. Other reasons for lack of adherence or noncompliance are numerous, possibly including that the patient forgot to take the medication, had symptoms that went away, wanted to save money, did not believe the drugs were effective, experienced unwanted side effects, may have feared addiction or the diversion of the drug to a third party unknown to the Caregiver, etc.

Many believe that physicians' fear of abuse potential leads to an under prescribing of certain abuse prone medications which offer great therapeutic value when properly used. This is no doubt true because until now the Caregiver has not been presented with an objective way of discerning potential abuse through simple examination.

Drug companies have attempted to curb abuse in a variety of ways. One method has been to “lock” the drug in a matrix so that the drug can only be used through an intended mode of administration. One such example is Flamel's “trigger lock” technology that seeks to prevent extraction of the drug from the dosage form by crushing or other traditional separation methods (see US 2008/0008659 A1; see also EP-A-1203209). Others have suggested the manufacture of tablets with great physical strength which are difficult to crush (US 2005/0031546 A1). Another drug specific approach has been to pair an opiate with an agonist in a sublingual dosage unit wherein the agonist is not sublingually absorbed in sufficient strength to impair the intended therapeutic use of the opiate (Lewis at al U.S. Pat. No. 4,582,835; see also Farrel US 2003/0026838 A1).

In sum, one can say that these attempts are primarily directed at preventing the user from using the drug in a way other than the intended modality by creating physical and or chemical barriers to such misuse. However, there is teaching of the use of dyes to show tampering of a dosage form by Thomas Gruber (U.S. Pat. No. 7,214,385 and assigned to an entity controlled by the instant inventors) and Chang et al (US Patent Application Publication No. 2004/0228802) and the use of a bittering agent to impart a bitter taste to an abuser by Oshlack et al. (US Patent Application Publication No. 20030064099) and Chang et al, the contents of each of which are incorporated by reference herein in their entireties.

### SUMMARY OF THE INVENTION

The present invention relates, inter alia, to a dosage form including a dose of at least one bioactive agent and a substance that can be readily confirmed by medical personnel to determine if the patient is compliant with taking the dosage form. The substance is at least one member selected from the group consisting of (a) a material incorporated in the dose in a manner that when the dosage form is properly administered is by itself or when combined with a reagent visible in the buccal, vaginal, nasal, rectal, aural, ophthalmic or ostomic cavity of a patient or an excretion of a patient in normal light or when exposed to a special frequency light source, (b) a material that is by itself not readily visible in the buccal, vaginal, nasal, rectal, aural, ophthalmic or ostomic cavity of a patient or an excretion of a patient in normal light but by itself or when combined with a reagent is readily visible when exposed to a special frequency light source, (c) a material that is by itself not readily visible in the buccal, vaginal, nasal, rectal, aural, ophthalmic or ostomic cavity of a patient or an excretion of a patient in normal light but when combined with a reagent is readily visible in normal light and (c) a material that is by itself not readily visible in the buccal, vaginal, nasal, rectal, aural, ophthalmic or ostomic cavity of a patient or an excretion of a patient in normal light but by itself or when combined with a reagent is readily detected by a non-visual detection method.

The present invention also relates to methods of using a Closed Loop Diagnostic System in order to determine if the patient is compliant with taking the dosage form and methods of promoting compliance with a risk evaluation and mitigation strategy.

### DETAILED DESCRIPTION OF THE INVENTION

The present invention is a methodology for effectively monitoring if a drug is taken as directed, i.e., if the user is taking the drug or other bioactive agent in compliance with a prescribed treatment plan.

All abuse of a drug first and foremost relates to the compliance of the user. Hence it is the determination of compliance which becomes the window to objectify non compliance which only then can result in abuse. Abuse, unless it results from elicit manufacture, starts with non-compliant use. That is, if a user/patient is not compliant with use of a drug, i.e., is not taking the drug as prescribed, the prescribed drug is available for non-compliant use by others; whereas if the user/patient is compliant with use of a drug, i.e., is taking the drug as prescribed, the prescribed drug is not available abuse by others. Therefore, the measure by caregivers of the compliant use by patients is an important method, e.g., through risk evaluation and mitigation strategies (REMS), for stopping abuse.

The present invention seeks to avoid abuse and non-compliance. However, the present invention functions in a way which is distinct from, and novel with respect to, the prior art. Rather than seeking to lock the drug in a dosage form for a particular use, a method which even if it works, allows no objective sign of abuse or compliance to the Caregiver, the present invention, inter alia, provides a dosage form and method for easily confirming compliant use of the product. Specifically, the present invention involves the use of a long acting staining or marking agent for use with an oral dosage form such that the mucosa is stained for a period to confirm use by the patient (or failure to use, as the case may be, or use prior to an emergency situation in which the use may in fact be abuse) of the dosage form as intended. When failure to use is discovered by healthcare professionals, such failure can be appropriately dealt with. In addition if the dosage unit here is crushed and snorted for a solid dosage form or dried and snorted for a liquid dosage form, in both cases a telltale nasal stain is also noted under some frequency of light.

This invention further relates to the use of a Trojan like agent (herein called a Trojan agent)—which may not be visible by itself under visible or normal light but is otherwise readily identifiable either at the site of administration (e.g. oral mucosa) or upon excretion through the body (i.e. urine, fecal matter, through the skin, etc.) and may react with a secondary external reagent to give a reaction which indicates the usage of the aforementioned bioactive either on a body surface, mucosal surface or in an excretion.

In one aspect of the present invention, an oral medication includes a dose of at least one bioactive agent and a substance incorporated in the dose in a manner that, when the oral medication is properly administered, leaves a stain or mark in a patient's mouth, by itself or when combined with a reagent, visible to a normal light or to a special light frequency source (such as but not limited to, ultraviolet or blacklight) in a patient's mouth in order for medical personnel to determine that the patient is in compliance with taking the medication, or, whether or not the oral medication is properly administered, leaves a stain or mark in a patient's mouth when combined with a reagent visible to a normal light, or whether or not the oral medication is properly administered, leaves a stain or mark in a patient's mouth, by itself or when combined with a reagent, visible to a special light frequency source in a patient's mouth in order for medical personnel to determine that the patient is in compliance with taking the medication.

Another aspect of the current invention relates to the use of a Trojan agent in a dosage form that is naturally excreted by the body, including through the skin as a method of determining compliance by the patient. Such agent may not be visible but is primarily selected such that its presence—followed by excretion by the body—can be readily confirmed more easily than traditional diagnostic testing. Like the use of staining agents described herein, the present invention may also use a Closed Loop Diagnostic System (CLDS) by which a specifically identifiable Trojan agent can be delivered together with the intended bioactive agent such that the subsequent confirmation of the presence of the Trojan agent indicates compliant use of the dosage form by the patient.

In another embodiment, the Trojan agent is excreted from the body through the skin. Garlic is widely understood to excrete in broken down dorm from the pores of the skin. In another embodiment the Trojan agent is excreted in the urine. In yet another embodiment the Staining Agent or Trojan agent is bound to the analyte of interest so that in the excretion of the bound material one gains insight as to the presence of the analyte.

In another embodiment the Staining Agent or Trojan agent is readily visible in the urine of a patient.

The present invention can take the form of a solid dosage form including a dose of at least one bioactive agent and a substance incorporated in the dose in a manner that, when the dosage form is properly administered, is, by itself or when combined with a reagent, visible to a normal light or to a special light source in the buccal, lingual, nasal, sublingual, vaginal, rectal, aural, ophthalmic or ostomic cavity or excretions of a patient in order for medical personnel to determine if the patient is being compliant with taking medication, or, whether or not the dosage form is properly administered, leaves a stain or mark in the buccal, lingual, nasal, sublingual, vaginal, rectal, aural, ophthalmic or ostomic cavity or excretions of a patient when combined with a reagent visible to a normal light, or whether or not the dosage form is properly administered, leaves a stain or mark in the buccal, lingual, nasal, sublingual, vaginal, rectal, aural, ophthalmic or ostomic cavity or excretions of a patient, by itself or when combined with a reagent, visible to a special light frequency source in a patient's mouth in order for medical personnel to determine that the patient is in compliance with taking the medication, wherein the substance is either coating the at least one bioactive agent or intermixed with the at least one bioactive agent in a delivering body, such as a capsule, or the absence of any evidence of a stain in any circumstance direct or indirect and under any frequency of light and thereby indicating that the patient did not in fact take the medication which dosage is therefore available for abuse.

It should be noted that the CLDS system uses a Trojan agent but that agent is not present in the completed bioactive dosage form because it may have bioactivity nor because it is a necessary excipient used for manufacturing, nor for coloring the bioactive in the sense of branding the bioactive (blue Viagra®, blue 10 mg. Valium® etc). It also should be noted that in at least one application the Trojan Agent is incorporated into the “granulation” of a solid dose form, the extruded mass of a sheet dose form, the liquid phase of a cast film or the solid or liquid phase of an inhaled volatilized dosage form, or the liquid base of a liquid dosage form or the flavoring agent such as flavor X®, or the liquid or particulate of our prior invention for ease of swallowing, or in some cases a coating of a solid dosage form.

When it is desired for the substance to be visible (by itself or when combined with a reagent) under normal light in the cavity to which the dose is properly administered or when exposed to a special frequency light source in the cavity to which the dose is administered, the substance is incorporated not only in an amount sufficient to be visible but also in a manner enabling the substance to adhere to the cavity (e.g., oral cavity). For example, if the dose has a controlled release coating, enteric coating or capsule, the substance should be coated over (on the outside) and/or mixed with the controlled release coating, enteric coating or capsule. It may additionally be coated on or mixed with the bioactive agent within the controlled release coating, enteric coating or capsule. In that manner, the substance is available to adhere to the cavity (e.g., oral cavity) as soon as it is administered.

In the case of using a substance that enables medical personnel to determine if the patient is compliant with taking the dosage form by examining the excretion of the patient, e.g., urine, the substance may also be a material that is by itself not readily visible in the excretion in normal light but by itself is readily visible when exposed to a special frequency light source or, when combined with a reagent, is readily visible under normal light or readily visible when exposed to a special frequency light source. Riboflavin, for example, has a yellow color in urine, which typically also has a yellow color for healthy, normally hydrated individuals, and thus Riboflavin can be difficult to see (i.e., not readily visible) in the urine under normal light. However, when exposed to light in the ultraviolet range (e.g., light from a “blacklight” source), Riboflavin in the urine is readily visible.

The substance may also be a material that is by itself not readily visible in the excretion in normal light but by itself or when combined with a reagent is readily detected by a non-visual detection method (e.g., garlic detected as an excretion through the pores).

The present invention can take the form of an oral medication comprising a dose of at least one bioactive agent and a substance that is excreted by the body such that the presence of the substance in an excretion of the body can be readily confirmed by medical personnel to determine if the patient is compliant with taking the oral medication, wherein the substance is at least one member selected from the group consisting of (a) a material incorporated in the dose in a manner that when the oral medication is properly administered is by itself or when combined with a reagent visible in normal light or when exposed to a special frequency light source, (b) a material that is by itself not readily visible in the excretion in normal light but by itself or when combined with a reagent is readily visible when exposed to a special frequency light source, (c) a material that is by itself not readily visible in the excretion in normal light but when combined with a reagent is readily visible in normal light and (d) a material that is by itself not readily visible in the excretion in normal light but by itself or when combined with a reagent is readily detected by a non-visual detection method.

The present invention can also take the form of a dosage form comprising a dose of at least one bioactive agent and a substance that can be readily confirmed by medical personnel to determine if the patient is compliant with taking the dosage form, wherein the substance is at least one member selected from the group consisting of (a) a material incorporated in the dose in a manner that when the dosage form is properly administered is by itself or when combined with a reagent visible in the buccal, vaginal, nasal, rectal, aural, ophthalmic or ostomic cavity of a patient or an excretion of a patient in normal light or when exposed to a special frequency light source, (b) a material that is by itself not readily visible in the buccal, vaginal, nasal, rectal, aural, ophthalmic or ostomic cavity of a patient or an excretion of a patient in normal light but by itself or when combined with a reagent is readily visible when exposed to a special frequency light source, (c) a material that is by itself not readily visible in the buccal, vaginal, nasal, rectal, aural, ophthalmic or ostomic cavity of a patient or an excretion of a patient in normal light but when combined with a reagent is readily visible in normal light and (d) a material that is by itself not readily visible in the buccal, vaginal, nasal, rectal, aural, ophthalmic or ostomic cavity of a patient or an excretion of a patient in normal light but by itself or when combined with a reagent is readily detected by a non-visual detection method.

As noted above, it may be beneficial to bind the bioactive agent to a staining agent or a Trojan agent. For example Glucose may be bound to another agent, for example a Staining Agent, to permit its identification. Take for example riboflavin as a staining agent. Glucose and riboflavin (Vitamin B 2) both are void of carboxylic acid groups. Both glucose and riboflavin have reactive hydroxy groups. Polysaccharides are prepared from the monosaccharides in the presence of phosphoric acid. For example, sucrose, a disaccharide can be prepared from glucose phosphate and another molecule of glucose or fructose. The linking group is an ether group which can be cleaved back to the two monosaccharides in the body. It is possible to react the glucose phosphate ester with riboflavin to hook the two molecules together through the —CH20H group present on the riboflavin molecule to produce an ether group with the glucose molecule. Alternatively, one may use the mesylate or tosylate ester of glucose to react with the acidic —CO—NH—CO— group in the riboflavin to attach the glucose molecule to the riboflavin, and this bond through the nitrogen would be stable and probably would not cleave under conditions in the body. This would allow the identification of the riboflavin in, for example, urine, to indicate the presence of glucose (see the examples discussed hereinafter).

The dosage form can be any solid dosage form currently existing or hereafter developed, e.g., capsule, tablet, film, sheet, quick dissolve solid, medicated lozenge, medicated lollipop, etc. The present invention may also be used with a liquid dosage form, including inter alia a solution or suspension of particles or nanoparticles or such other liquid dosage form as may be developed in the future.

The bioactive agents can include, but are not limited to ace-inhibitors, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, pain medications (short and long acting), parasympathomimetics, prostaglandins, anti-prostaglandins, psychotherapeutic agents, respiratory agents, sedatives, smoking cessation aids, sympatholytics, sympathomimetics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythropoietic drugs, anti-asthmatics, cough suppressants, mucolytics, DNA and genetic modifying drugs, and combinations thereof. The analgesics can include opiates and opiate derivatives, such as oxycodone (available as Oxycontin™) buprenorphine, methadone, ibuprofen, aspirin combinations, acetaminophen, and combinations thereof that may optionally include caffeine. The analgesics can include any pain medication. These also include drug pre dosage units as, for example, those volatilized and then inhaled. These mark the nasal mucosa. This invention can also include tobacco, nicotine and tobacco and nicotine derived liquids, some of which might be volatilized into a vapor.

The present invention is especially effective for use in connection with a bioactive agent that is adjudged by the health care provider to be especially subject to abuse by the patent or general populace, especially bioactive agents prescribed for a limited period of time. This category is mainly pain directed drugs but is not limited thereto. Such bioactive agents include, but are not limited to buprenorphine (Buprenex), butorphanol (Stadol), codeine (Hydrocodone), hydromorphone (Dilaudid, Dilaudid-5, Dilaudid-HP, Hydrostat IR), levorphanol (Levo-Dromoran), meperidine (Demerol), methadone (Dolophine, Methadose), morphine (Astramorph PF, AVINZA, Duramorph, Kadian, M S Contin, MSIR, Oramorph SR, Rescudose, Roxanol), nalbuphine (Nubain), oxycodone (OxyContin, Roxicodone), oxymorphone (Numorphan), pentazocine (Talwin), propoxyphene (Cotanal-65, Darvon), tramadol (Ultram), tramadol and acetaminophen combination (Ultracet), butalbital, acetaminophen, and caffeine combination (Femcet, Fioricet, Esgic, Esgic-Plus), butalbital, aspirin, and caffeine combination (Fiorinal), butalbital, acetaminophen, caffeine, and codeine combination (Fioricet with Codeine), hydrocodone and ibuprofen combination (Hydrostal IR, Vicoprofen), pentazocine/naloxone (Talwin NX), acetaminophen and codeine combination (Capital with Codeine, Margesic #3, Phenaphen with Codeine, Tylenol with Codeine), dihydrocodeine, acetaminophen, and caffeine combination (DHCplus), hydrocodone and acetaminophen combination (Allay, Anexsia 5/500, Anexsia 7.5/650, Dolacet, Dolagesic, Duocet, Hycomed, Hydrocet, Hydrogesic, HY-PHEN, Lorcet 10/650, Lorcet-HD, Lortab, Panacet 5/500, Panlor, Stagesic, T-Gesic, Ugesic, Vicodin, Zydone), oxycodone and acetaminophen combination (Endocet, Percocet, Roxicet, Roxilox, Tylox), pentazocine and acetaminophen combination (Talacen), propoxyphene and acetaminophen combination (Darvocet-N 50, Darvocet-N 100, E-Lor, Propacet 100), aspirin, caffeine, and dihydrocodeine combination (Synalgos-DC), aspirin and codeine combination (Empirin with Codeine), hydrocodone and aspirin combination (Damason-P, Lortab ASA, Panasal 5/500), oxycodone and aspirin combination (Endodan, Percodan, Percodan-Demi, Roxiprin), pentazocine and aspirin combination (Talwin Compound), and propoxyphene, aspirin, and caffeine combination (Darvon Compound 65, PC-Cap, Propoxyphene Compound 65)

The “staining agent” in the dosage form is visible to a normal or special frequency light source, e.g., an ultraviolet or black light source, etc. in order for medical personnel to determine if the patient is being compliant with taking medication. The substance can be a dye/stain (selected for being edible and also having a half life as a mucosal stain, e.g., a half buccal life, of, e.g., 6-24 hours (or shorter or longer duration)), and/or a substance that is a dye/stain, or a substance other than a dye/stain, e.g., a composition that shows under certain light. As used herein, “staining agent” is used to mean a substance, whether a dye or stain, that is visible in normal light or when exposed to a special frequency light source. The substance can be incorporated into any oral or other dosage unit. The stain from the substance is then visible in the patient's mouth, e.g., on the patients tongue, nasal passages, sublingual space, buccal space or a patient's vaginal, rectal, aural, ophthalmic or ostomic cavity for a predetermined time period. The term “stain” is also used herein with the same meaning as “staining agent” or “dye” or “marking agent”.

“Trojan Agent” as used herein means a substance or agent which can be readily identified at either the surface of administration of the dosage form, or following excretion by the body either directly or through a specific light frequency or through use of a reagent system. The Trojan Agent is selected to be readily identifiable and it generally but not always will have no significant direct therapeutic effect It is possible that the Trojan Agent is identified using reagents or other conventional methods of chemical identification which are well suited to use by the typical caregiver (i.e. preferably inexpensive, accurate and quick testing). It is part of the CLDS system. The Trojan agent while used for compliance may also show abuse such, but not limited to, as nasal snorting by itself or through a reagent and when viewed under specific light or light frequencies. It showed also be noted that the same procedure that shows compliance in a patient may show abuse in an unauthorized user even if used in a pharmaceutically proper manner.

For example, a Trojan agent may be included in a dosage form wherein the Trojan agent is excreted through the skin. Using a simple reagent paper, the Caregiver confirms that the patient is excreting the Trojan agent, thereby providing an objective indicator that the dosage form has been used. The basic principle is to include a medically safe and accepted staining or Trojan agent in the dosage form, especially (but not limited to) those dosage forms that have a residence time of 10 seconds or more in the mammalian oral, vaginal, rectal, aural, ophthalmic or ostomic cavity. It is possible to use this methodology with conventionally administered tablets, which have a much shorter residence time, as is successfully demonstrated in the examples below, but is easier to employ the system where the residence time is greater. In the case of a Trojan agent, residence time may be important where one wants to identify the Trojan agent at the site of administration. However, residence time will not be important where the Trojan agent is identified in bodily excretions.

It is preferable that the staining agent or Trojan agent be non-toxic, such that the staining agent does not cause damage in the event that the dosage form is misused by snorting or mainlining. It is an express intention of this invention that in certain embodiments, the staining agent may stain the nasal mucosa in the event that the dosage form is crushed and snorted or volatilized thereby demonstrating such misuse. Similarly, the Trojan agent may coat the nasal mucosa where a dosage form has been crushed and snorted, and the presence of the Trojan agent on the mucosal surface may indicate abuse.

The Trojan agent may be a non-visual Trojan agent such as, but not limited to micro radio tag particles and low level radiation markers.

It may be desirable to include a bittering agent which may irritate the user when the dosage form is snorted but does not bother the patient when the dosage form is properly used. A bittering agent can be used in conjunction with one or more Staining agent and/or Trojan agents.

It is preferable that the staining effect of the staining agent have a controlled, limited duration. Ideally, the staining effect will correspond to the expected use of the drug. For example, an agent that is administered once daily should have a staining effect that lasts, when used as directed, approximately 24 to 30 hours. Similarly with the Trojan agent, it is desirable to have a certain half life in the body's metabolism, such that the presence (or non-presence) of the Trojan agent is a useful proxy for intended drug compliance.

When the mucosa is effectively stained by the present method, the presence of the stain can be confirmed in the normal course by healthcare personnel. While, admittedly a temporary stain is not considered desirable, it is contemplated that particularly for powerful abuse prone drugs, that the patient will not be bothered by such staining. For example, a patient who is prescribed the fentanyl lollipop will presumably not be bothered by a temporary staining of the buccal cavity associated with its use. It will also give health care personal a double check that the agent has been given and taken. This adds an additional layer of safety. Additionally, it is possible to use a Staining Agent that is not visible in natural light. While such agent will stain in special light, it will not be visible to the patient in natural light and this embodiment may be useful for drugs where the patient does not wish to be visibly marked by the administration thereof.

Stain concentration as a percentage of the dosage form composition, for a given dye agent, will control the speed during which the staining occurs, as well as the duration of the stain on the target area. The total amount of the marking or staining agent is must be sufficient to mark or stain the cavity or excretion, especially for very small dose forms.

The necessary stain concentration gradient depends on residence time for proper use of the dosage form. These same principles apply in part to the use of a Trojan agent as respect mucosal surfaces.

It may be desirable to that the dosage form not stain immediately, but rather require the intended residence time for the product in order to stain as this is also in keeping with the normal dissolution pattern of the dosage unit in compliant use. As a result, the stain confirms that the dosage form has been used as intended, and not inserted briefly in for example the mouth and then removed for unintended use. The same principles apply in part to the use of a Trojan agent as respect mucosal surfaces.

It is also contemplated that two (or more) staining agents may be used in a single dosage form. These staining agents may be mixed equally throughout the dosage form, or separated and or segregated in different areas of the dosage form. For example, the outer portion, or but not limited to coating, of a tablet may contain one staining agent of a certain color, and the inner portion may contain a second staining agent of a different color. The effect of the use of two different staining agents may be to further confirm that the entire dosage form as been used as intended. The absence of the staining effect caused by the exposure to the two successive staining agents indicates that the dosage form as not been completely used as intended, and may have been partially diverted for potential misuse.

It may be desirable to combine in a single dosage form one staining agent that is visible in normal might with a second agent that is visible only in special light. For example in one non limitative embodiment, one agent may serve to visibly stain the mucosa while a second agent is visible in excretions (e.g. urine) when under special light.

One may also combine one or more staining or Trojan agents in the same dosage form, again, for multiple objective indicators of compliance (or non compliance) that can be confirmed by the Caregiver.

The staining agent or Trojan agent may be placed in an outer coating on the dosage form but the preferred embodiment is throughout the dosage form for compliance reasons. Tablet coating technologies are understood in the industry. Coatings may also be used without a staining agent to prevent a staining effect from occurring on the fingers of individuals handling the dosage form. A bittering agent may be used in conjunction with any of these combinations.

The Staining agent or Trojan agent may be combined with, or attached to an agent that adheres to a mucosal surface. This allows the dose to be retained on the mucosal surface, e.g., on the tongue, long enough for the dye or stain to stain the mucosal surface.

The staining agent may also be visible (in natural or special light) in the body's excretions including inter alia urine and/or fecal matter or sweat. Alternatively staining agents may purposefully be selected which blend with the natural colors of urine or fecal matter so as not to create concerns for the patient. It may be further desirable to use a staining agent that blends with the natural color in visible light, but is visible in special frequency light. For example, as is demonstrated in the examples, Riboflavin does not create a material change in the color of urine under normal light, but the urine of the compliant patient will be fluorescent under black light. Thus, simple, immediate visual inspection by the caregiver of urine and or fecal matter can be conducted (with or without special light) to confirm compliance—that the drug has been taken and not diverted.

Various embodiments of the current invention may be incorporated into risk evaluation and mitigation strategies (REMS) as are required by the United States Food and Drug Agency. One of the components of REMS is to have the physician or health care provider search for signs of abuse. With the current invention, the physician or other individual can be told to look for various objective signs of compliant and non-compliant use. This is very important because the Caregiver must be able to objectify his opinions of a particular patients use or abuse. Presently REMS, in large part confer the obligation to the Caregiver but do not convey the tools to the Caregiver. This methodology is such a tool. Hence this objective modality is a critical improvement to the REMS system currently in use.

As an example of a Staining Agent, but without limitation, the stain Gentian Violet in 0.0001% to 20% concentration (measured by percentage mass of the entire dosage form) can be used as a substance that is visible to a normal light or to a special light source in the mammalian oral, vaginal, rectal, nasal, aural, ophthalmic or ostomic cavity in order for medical personnel to determine if the patient is being compliant with taking medication. This material is understood to be safe for all forms of delivery in the mammal (see Piatt and Bergeson, “Gentian Violet Toxicity” in Clinical Pediatrics, Vol. 31, No. 12, 756-757 (1992), addressing safety of Gentian Violet for treatment of thrush in human infants). It leaves behind a significant blue stain on the cavity in which it is placed thereby giving the health care provider ready knowledge of compliance and use as provided above.

In abuse and medical studies, the material has been safely used intravenously although we would consider this an abuse of the drug and the drug could be detected in the blood stream. It is understood that gentian violet when injected into a mammal may be visible through the skin (see Churchman and Herz, “The toxicity of Gentian Violet and its fate in the Animal Body” in J Exp Med., 18 (5): 579 (1913)). Other staining agents may similarly be visible when injected. Such staining agents may be visible through the skin, or when extracted as in an ordinary blood test under normal and/or special light. This may also serve as a useful objective indicator for use in connection with REMS. Trojan agents of the CLDS system likewise function as objective indicators in connection with REMS.

It may be desirable for a drug company to approve multiple staining agents and/or Trojan agents for a given product and rotate their use as part of a strategy to defeat work-arounds by abusers.

Other examples of substances visible in mammalian oral, vaginal, rectal, nasal, aural, ophthalmic or ostomic cavity, e.g., having the desired staining effect, are described, without limitation below.

The colored triarylmethanes of Formula I and Formula II below are useful in the practice of the invention:

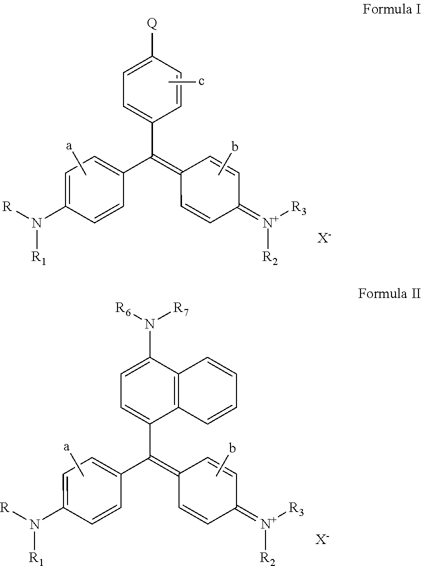

wherein R, R₁, R₂ and R₃ are independently selected from hydrogen, C₁-C₆-alkyl, substituted C₁-C₆-alkyl, C₃-C₆-alkenyl, C₃-C₆-cycloalkyl, and aryl; a, b and c represent hydrogen or are independently selected from C₁-C₃-alkyl and halogen; Q is selected from hydrogen, C₁-C₆-alkyl, C₁-C₆-alkoxy, halogen and —N(R₄)R₅, wherein R₄ and R₅ are independently selected from hydrogen, C₁-C₆-alkyl, substituted C₁-C₆-alkyl, C₃-C₆-alkenyl, C₃-C₆-cycloalkyl and aryl; R₆ is selected from C₁-C₆-alkyl, C₃-C₆-cycloalkyl and aryl; R₇ is hydrogen or C₁-C₆-alkyl; X⁻ is an anion selected from Cl⁻, Br⁻, I⁻, CH₃CO₂⁻, HSO⁻₄ᵢ,

$$\frac{{ZnCl}_{4}^{=}}{2} ,$$

and the like.

Compounds of the Formulae I and II range in color from red to green. In general, compounds of the Formula I wherein Q is an —N(R₄)R₅ group are red to violet, usually violet. Compounds of the Formula I wherein Q is hydrogen, or is a group selected from hydrogen, halogen, C₁-C₆-alkyl and C₁-C₆-alkoxy, usually range in color from bluish-green to green and compounds of Formula II are usually blue.

Some of the preferred compounds are the violet compounds of Formula I, wherein Q is —N—(R₄)R₅, i.e., compounds of the following Formula III:

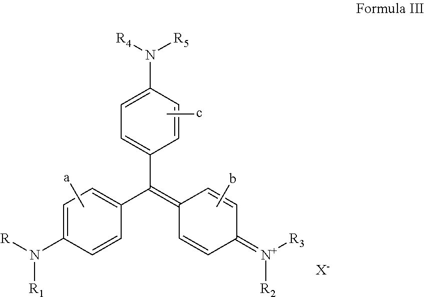

wherein R, R₁, R₂, R₃, R₄ and R₅ are independently selected from hydrogen, methyl and ethyl; a, b and c are hydrogen; X⁻ is Cl⁻. The most preferred compound of Formula III is the violet compound wherein R, R₁, R₂, R₃, R₄ and R₅ are methyl and X⁻ is Cl⁻, i.e., compounds of the following Formula IV:

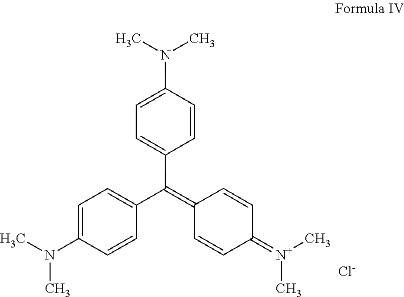

In the Colour Index International, 3^{rd} Edition (published by The Society of Dyers and Colourists with the American Association of Textile Chemists and Colorists), this compound has been assigned The Colour Index Generic Name, Basic Violet 3, and the Colour Index Constitution Number, C.I. 42555.

For the sake of clarity, it should be mentioned that the resonance structure provided above as Formula III is only one of three possible structures, the other two having the structures in the following Formula V and VI:

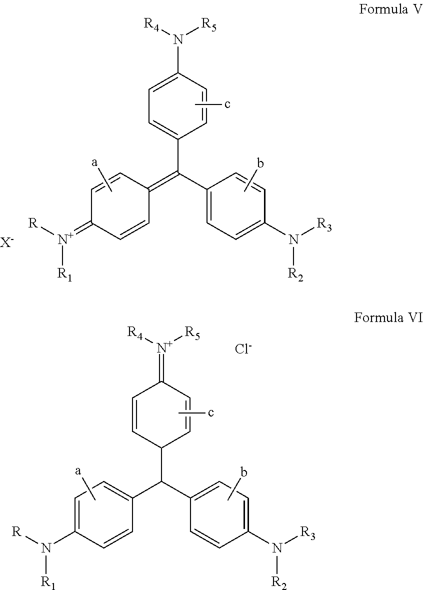

For further clarity, it should also be mentioned that two other structures have been traditionally used for compounds of the Formula III and triarylmethane type compounds in general, as the following Formula VII and Formula VIII:

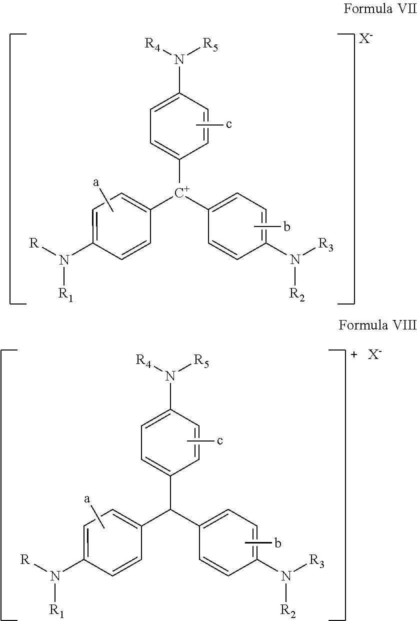

The terms “C₁-C₃-alkyl” and “C₁-C₆-alkyl” are used above to denote an aliphatic hydrocarbon radical that contains one to three and one to six carbon atoms, respectively, and the hydrocarbon radicals are either a straight chain or a branched chain.

The term “substituted C₁-C₆-alkyl” is used to denote a C₁-C₆-alkyl radical substituted with one or two groups selected from the following: halogen, hydroxy, cyano, carbonyl, aryl, C₃-C₆-cycloalkyl, succinimido, glutarimido, phthalimido, phthalimidino, 2-pyrrolidino, o-benzoicsulfimido, heteroaryl, C₁-C₆-alkoxy; C₁-C₆-alkylthio, C₁-C₆-alkylsulfonyl, heteroarylthio, C₁-C₆-alkanoyloxy, aryloxy, arythio and arylsulfonyl.

The terms “C₃-C₆-alkenyl” denotes a straight or branched chain hydrocarbon radical containing three to six carbon atoms and at least one carbon-carbon double bond.

The term “C₃-C₆-cycloalkyl” denotes a saturated cycloaliphatic radical containing three to six carbon radicals.

The terms “C₁-C₆-alkoxy”, “C₁-C₆-alkylthio”, “C₁-C₆-alkylsulfonyl” and “C₁-C₆-alkanoyloxy” denote the following structures, respectively —O—C₁-C₆-alkyl, —SC₁-C₆-alkyl, —O₂SC₁-C₆-alkyl and —OCOC₁-C₆-alkyl.

In the terms “aryl,” “arythio,” “aryloxy” and “arylsulfonyl,” the aryl groups are selected from a phenyl group and phenyl substituted with one or two groups selected from C₁-C₆-alkyl, C₁-C₆-alkoxy, halogen and hydroxy.

In the terms “heteroaryl” and “heteroarylthio” the heteroaryl groups or heteroaryl portions of the groups are mono or bicyclo heteroaromatic radicals containing at least one hetero atom selected from the group consisting of oxygen, sulfur and nitrogen, or a combination of these, to complete the heteroaromatic ring. Examples of suitable heteroaromatic groups include: furyl, thienyl, thiazolyl, benzothiazolyl, isothiazolyl, pyrazolyl, pyrolyl, pyrimidinyl and triazolyl and such groups substituted with one or two groups selected from C₁-C₆-alkyl, C₁-C₆-alkoxy, C₃-C₆-cycloalkyl, C₁-C₆-alkylthio, aryl, arylthio, aryloxy and halogen.

The term “halogen” is used to include fluorine, chlorine, bromine and iodine.

The term “carbamyl” is used to represent the group having the formula CON(R₈R₉), wherein R₈ and R₉ are independently selected from hydrogen, C₁-C₆-alkyl and aryl.

Triarylmethane colorants of Formula I and Formula II are generally well-known and many are listed in the Colour Index International, 3^{rd}. Ed., Vol. 4, pages 4380-4407. Many references are also provided there to synthetic methods for preparing the compounds. Also, provided by the Colour Index International, 3^{rd} Ed., Vol. 5 are Colour Index Generic Names (C.I. Generic Names) and Colour Index Constitution Numbers (C.I. Constitution Numbers) for many specific structures. Colour Index International, 3^{rd}. Ed., Vol. 5, also provides a list of manufacturers/suppliers for each C.I. Generic Name.

Examples 1-15 in Table I below provide several specific compounds included in Formula I or Formula II above, which are useful in the practice of the present invention. Also, the C.I. Generic Names and C.I. Constitution Numbers are provided for the colored compounds of Examples 1-15.

TABLE 1
Typical Useful Triphenylmethane Dye

| | | | C.I. |
|---|---|---|---|
| Example | | C.I. | Comparison |
| Number | Structure | Generic Name | Number |
| 1 | Formula IV | Basic Violet 3 | C.I. 42555 |
| 2 | Formula IX below | Basic Violet 4 | C.I. 42600 |
| 3 | Formula X below | Basic Violet 2 | C.I. 42520 |
| 4 | Formula XI below | Basic Violet 1 | C.I. 42535 |
| 5 | Formula XII below | Basic Violet 14 | C.I. 42510 |
| 6 | Formula XIII below | Basic Red 9 | C.I. 42500 |
| 7 | Formula XIV below | Basic Violet 23 | C.I. 42557 |
| 8 | Formula XV below | Basic Green 1 | C.I. 42040 |
| 9 | Formula XVI below | Basic Green 4 | C.I. 42000 |
| 10 | Formula XVII below | Basic Blue 1 | C.I. 42025 |
| 11 | Formula XVIII below | Basic Blue 8 | C.I. 42563 |
| 12 | Formula XIX below | Basic Blue 11 | C.I. 44040 |
| 13 | Formula XX below | Basic Blue 7 | C.I. 42595 |
| 14 | Formula XXI below | Basic Blue 26 | C.I. 44045 |
| 15 | Formula XXII below | Basic Blue 15 | C.I. 44085 |

Formula IX 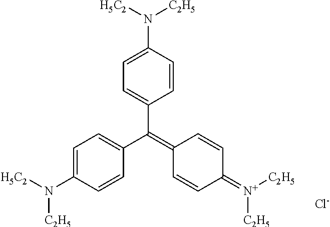
Formula X 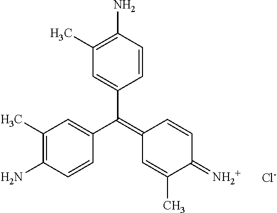
Formula XI 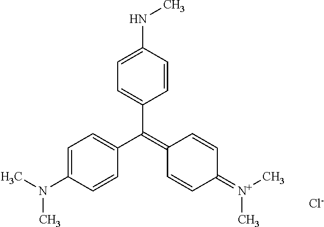
Formula XII 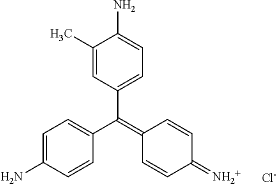
Formula XIII 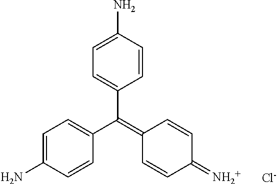
Formula XIV 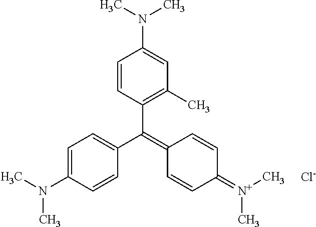
Formula XV 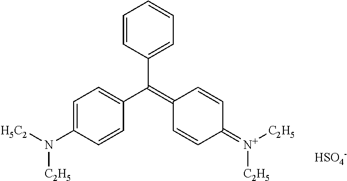
Formula XVI 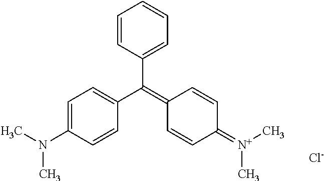
Formula XVII 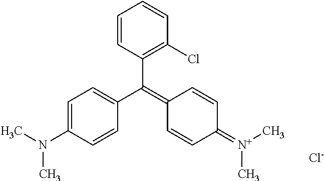
Formula XVIII 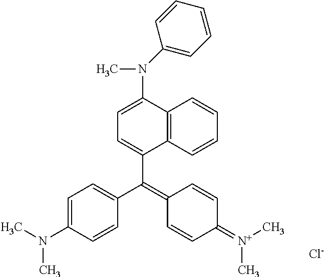
Formula XIX 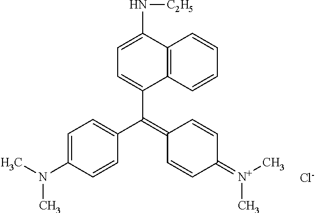
Formula XX 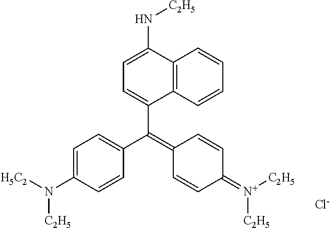
Formula XXI 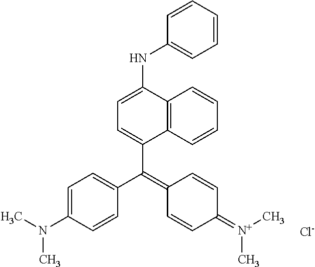
Formula XXII

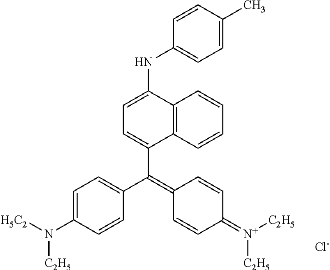

The above discussion of Gentian Violet Powder does not limit the range of staining agents that may be employed. Preferably, a staining agent that is GRAS will be employed or another agent with a well understood safety and toxicological profile. Various GRAS materials, including without limitation riboflavin, Talc, and other agents which are visible under specific light and may be suitable. Liquid dosage Units.

The same basic materials and principles which are used in some of the examples can apply to a liquid dosage unit. In that case any color they have in the liquid state can be additive to normal food dyes or used in place of same. Thereby using this system in liquid dosage units will confirm the same benefits to the Caregiver as expressed in solid dosage units. The liquid may be water but may also be modified water such as one of the inventor's past patents (U.S. Pat. No. 6,337,083, the contents of which are incorporated herein by reference) which is a modified liquid which eases the swallowing of a particulate suspension or a solution. It is also contemplated that this system could be incorporated via flavor systems used by the compounding Pharmacist, such as the flavoring product sold as Flavor X®. The liquid system is very important because in the future more drugs which are potentially abused may be in a liquid phase. Should the abuser sublime off the liquid for snorting or otherwise extract the drug, he is still left with the nasal staining and the urinary or fecal staining as described herein. In connection with this it should be of note that this CLDS system may also be used with emerging technologies which vaporize or atomize a drug which is then taken through the mouth and nose or alternatively through the nose alone. This system will make it possible to confirm compliance.

There follow some examples. In the following examples, Gentian Violet Powder HARLECO Certified Biological Stain was acquired from Gallade Chemical. Riboflavin was obtained from Colorcon.

### Example A

9.76 grams of Domino's confectionary sugar were placed in a crucible together with 0.24 grams of Gentian Violet powder. This blend was mixed thoroughly to create a 2.4% Gentian Violet concentration “master batch”.

Four TUMS® Ultra Strength 1000 tablets were pulverized using a mortar and pestle. Five drops of water were added to the pulverized TUMS® tablets and the blend was mixed thoroughly together.

9.6 grams of the crushed, wetted TUMS® tablets as described above were combined with 0.4 gram of the 2.4% Gentian Violet “master batch.” This blend was mixed thoroughly to make concentration of 0.096% Gentian Violet. 0.5 grams of this mix was placed into a single tablet press to make tablet. Eight more such tablets were made in the same fashion.

### Example B

The method of Example A was repeated with the use of a lower concentration of 2.4% Gentian Violet “master batch” to make eight tablets with a final concentration of 0.048% Gentian Violet.

### Example C

A healthy male volunteer placed the tablet of Example A in the buccal cavity and allowed the tablet to dissolve in his buccal cavity over a period of twenty five minutes. A blue stain resulted in the buccal cavity. This blue buccal stain was not visible when the subject conversed or ate. The stain lasted for approximately thirty hours, and disappeared without any permanent effect.

### Example D

A healthy male volunteer placed the tablet of Example B in the buccal cavity and allowed the tablet to dissolve in his buccal cavity over a period of twenty five minutes. A blue stain resulted in the buccal cavity. This blue buccal stain was not visible when the subject conversed or ate. The stain lasted for approximately six hours, and disappeared without any permanent effect.

### Example E

Four TUMS® Ultra Strength 1000 were crushed using mortar and pestle. Five drops of water where added to the crushed TUMS®tablets and they were mixed thoroughly. Then 0.1 gram of Gentian Violet was added to 1.9 gram wetted crushed TUMS®. The composition was mixed again, and then tablets were pressed (approximate total mass of 650 mg for each tablet) using a single tablet press, resulting in tablet with a concentration of 5%.

### Example F

A healthy male volunteer placed the a tablet made in Example E on his tongue and swallowed the tablet after approximately one second, using the same procedure that one typically uses for swallowing a conventional tablet. This administration resulted in a stain on the tongue that lasted over twelve hours despite the taking of routine meals and snacks as well as a tooth brushing.

### Example G

Using the same method as in Example E, tablets containing 10% Gentian Violet were made. A health male volunteer took one such tablet using the same procedure as Example F. A stain was visible on his tongue in normal light for over twenty seven hours, despite the taking of routine meals, snacks and tooth brushing.

### Example H

A healthy male volunteer took a tablet made in accordance with Example A and crushed the tablet. He then “snorted” the resulting powder. The result was to stain the nasal mucosa of the volunteer.

### Example I

Tums® Ultra Strength 1000 tablets were crushed. Then, for each batch using fifteen drops of water, the following concentrations were made: (a) 9.75 grams of Tums powder together with 0.250 grams of Riboflavin, (b) 9.8 grams of Tums powder together with 0.2 grams of Riboflavin and (c) 9.85 grams of Tums powder together with 0.15 grams of Riboflavin. Then, tablets were made at both 1.5 gram weight and 500 mg weight using the above mixes. Accordingly, the 1.5 gram tablets contained (d) 37.5 mg of riboflavin, (e) 30 mg of riboflavin and (f) 22.5 mg of riboflavin. The 500 mg tablets contained (g) 12.5 mg of riboflavin, (h) 10 mg of riboflavin and (i) 7.5 mg of riboflavin.

### Example J

A healthy male volunteer took the 500 mg tablet containing 12.5 mg of riboflavin prepared as in Example H, and allowed the tablet to dissolve in his buccal cavity. The tablet left no visible stain. The mouth was then illuminated in a dark room using a “Portable Six Inch Blacklight” sourced from Amazon.com. The area of the buccal cavity where the tablet was administered was clearly visible using the blacklight.

### Example K

A healthy male volunteer took the 1.5 gram tablet containing 22.5 mg of riboflavin prepared as in Example H.

The volunteer chewed the tablet fully in his mouth, swallowing the resulting slurry of dissolved tablet. Using a Portable Six Inch Blacklight, the volunteer's mouth was inspected as the following time intervals with the following observations:

| Hours after chewing and | |
|---|---|
| swallowing tablet | Observations |
| 0 hours | mouth and tongue bright yellow under normal |
| | light |
| 3 hours | yellow color slightly visible under normal light |
| | and very visible under black light |
| 6 hours | yellow tint only visible under black light after |
| | eating lunch on tongue |
| 9 hours | yellow tint visible under black light on tongue |
| 12 hours | tint visible under black light after dinner on |
| | tongue |
| 15 hours | yellow tint still visible on tongue under black |
| | light |
| 24 hours | after drinking coffee, yellow tint still visible on |
| | tongue around the edges and much more |
| | visible under the tongue with black light |

During the same administration, the volunteer's urine was examined, again, using a Portable Six Inch Blacklight (Amazon).

| Hours after chewing | |
|---|---|
| and swallowing tablet | Observations |
| 3 hours | bright psychedelic yellow tint instantly visible in |
| | drops of urine on a white surface. |
| 12 hours | bright psychedelic yellow tint instantly visible in |
| | drops of urine on a white surface. |
| 24 hours | under black light, after urine sample dries out a |
| | yellow tint can be detected |

At no time during the foregoing testing did the healthy volunteer perceive his urine as looking unnatural in natural light.

### Example L

The tablet containing 10 mg of riboflavin prepared in Example J was chewed and swallowed by a healthy male volunteer. H is urine was then examined with Portable Six Inch Blacklight (Amazon) with the following observations.

| Hours after chewing and | | |
|---|---|---|
| swallowing tablet | | Observations |
| 1 | hour | Very visible |
| 3 | hours | Very visible |
| 8 | hours | Faintly visible |
| Thirteen | hours | Not visible |

### Example M

A liquid was prepared using 200 g of water together with 20 mg of riboflavin. A healthy male volunteer drank the liquid and his urine was examined with a Six Inch Portable Blacklight. The urine was strongly visible under the blacklight for over forty hours.

### Example N

A healthy male volunteer purchased a pack of Meijer High Potency Reduced Odor Garlic tablets. As an example of a Trojan agent, the volunteer consumed three tablets of the product. His skin had a noticeable garlic odor six hours after the administration.

What is claimed is:

1. An oral medication comprising a dose of at least one bioactive agent and a substance incorporated in the dose in a manner that, when the oral medication is properly administered, leaves a stain or mark in a patient's mouth, by itself or when combined with a reagent, visible in normal light or when exposed to a special frequency light source in order for medical personnel to determine if a person is in compliance with taking the medication.

2. The oral medication according to claim 1, wherein the substance is visible for a predetermined time period in the patient's mouth.

3. The oral medication according to claim 1, wherein the substance is Gentian Violet.

4. The oral medication according to claim 1, wherein the substance riboflavin.

5. The oral medication according to claim 1, wherein the substance is a colored triarylmethanes of the following Formula I or Formula II: 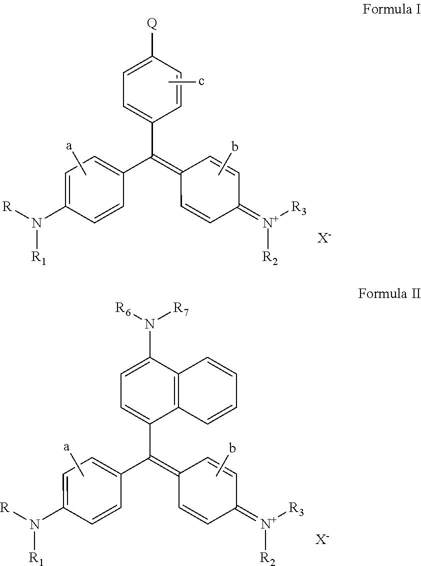
wherein R, R₁, R₂ and R₃ are independently selected from the group consisting of hydrogen, C₁-C₆-alkyl, substituted C₁-C₆-alkyl, C₃-C₆-alkenyl, C₃-C₆-cycloalkyl, and aryl; a, b and c represent hydrogen or are independently selected from the group consisting of C₁-C₃-alkyl and halogen; Q is selected from the group consisting of hydrogen, C₁-C₆-alkyl, C₁-C₆-alkoxy, halogen and —N(R₄)R₅, wherein R₄ and R₅ are independently selected from the group consisting of hydrogen, C₁-C₆-alkyl, substituted C₁-C₆-alkyl, C₃-C₆-alkenyl, C₃-C₆-cycloalkyl and aryl; R₆ is selected from the group consisting of C₁-C₆-alkyl, C₃-C₆-cycloalkyl and aryl; R₇ is hydrogen or C₁-C₆-alkyl; and X⁻ is an anion.

6. The oral medication according to claim 4, wherein the anion is selected from the group consisting of Cl⁻, Br⁻, I⁻, CH₃CO₂⁻, HSO⁻₄ᵢ,
$$\frac{{ZnCl}_{4}^{=}}{2} .$$

7. The oral medication according to claim 1, wherein the dose is in liquid form.

8. The oral medication according to claim 1, wherein the dose is in solid form.

9. The oral medication according to claim 8, further comprising an agent that adheres the dose to a surface of the buccal cavity for a period of time.

10. The oral medication according to claim 1, wherein the dose is in the form of an extruded sheet.

11. The oral medication according to claim 1, wherein the dose is in the form of a cast film.

12. The oral medication according to claim 1, wherein the dose is in the form of a lollipop or lozenge.

13. A dosage form comprising a dose of at least one bioactive agent and a substance incorporated in the dose in a manner that, when the dosage form is properly administered, is, by itself or when combined with a reagent, visible to a normal light or to a special light source in the buccal, vaginal, nasal, rectal, aural, ophthalmic or ostomic cavity of a patient or excretion of a patient in order for medical personnel to determine if the patient is being compliant with taking the dosage form, wherein the substance is either coating the at least one bioactive agent or intermixed with the at least one bioactive agent in a delivering body.

14. The dosage form according to claim 13, wherein the substance is coated in an outer surface of the at least one bioactive agent.

15. The dosage form according to claim 14, wherein the substance is intermixed with the at least one bioactive agent in the delivering body.

16. The dosage form according to claim 13, wherein the delivering body is a capsule.

17. The dosage form according to claim 13, wherein the delivering body is an extruded sheet.

18. The dosage form according to claim 13, wherein the delivering body is a cast film.

19. The dosage form according to claim 13, wherein the delivering body is a liquid.

20. The dosage form according to claim 13, wherein the substance is Gentian Violet.

21. The dosage form according to claim 13, wherein the substance is Riboflavin.

22. The oral medication according to claim 13, wherein the substance is a colored triarylmethanes of the following Formula I or Formula II: 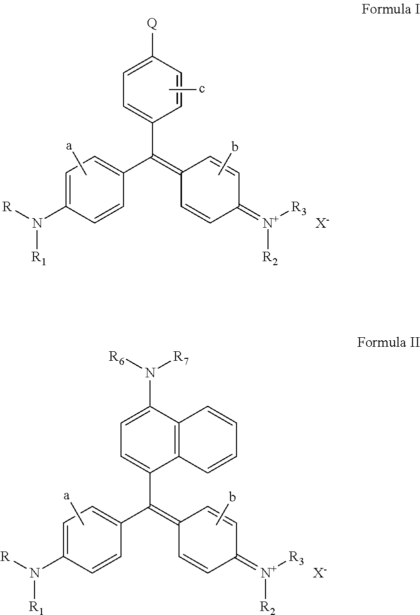
wherein R, R₁, R₂ and R₃ are independently selected from the group consisting of hydrogen, C₁-C₆-alkyl, substituted C₁-C₆-alkyl, C₃-C₆-alkenyl, C₃-C₆-cycloalkyl, and aryl; a, b and c represent hydrogen or are independently selected from the group consisting of C₁-C₃-alkyl and halogen; Q is selected from the group consisting of hydrogen, C₁-C₆-alkyl, C₁-C₆-alkoxy, halogen and —N(R₄)R₅, wherein R₄ and R₅ are independently selected from the group consisting of hydrogen, C₁-C₆-alkyl, substituted C₁-C₆-alkyl, C₃-C₆-alkenyl, C₃-C₆-cycloalkyl and aryl; R₆ is selected from the group consisting of C₁-C₆-alkyl, C₃-C₆-cycloalkyl and aryl; R₇ is hydrogen or C₁-C₆-alkyl; and X⁻ is an anion.

23. The dosage form according to claim 21, wherein the anion is selected from the group consisting of Cl⁻, Br⁻, I⁻, CH₃CO₂⁻, HSO⁻₄ᵢ,
$$\frac{{ZnCl}_{4}^{=}}{2} .$$

24. The dosage form according to claim 13, wherein the delivering body is in solid form.

25. The dosage form according to claim 24, further comprising an agent that adheres the delivering body to a surface of the buccal cavity for a period of time.

26. The dose form of claim 13, wherein the dose form is in a solid or liquid form and then normally volatilized or atomized for inhalation through the respiratory system, including the nasal mucosa.

27. An oral medication comprising a dose of at least one bioactive agent and a substance that is excreted by the body such that the presence of the substance in an excretion of the body can be readily confirmed by medical personnel to determine if the patient is compliant with taking the oral medication, wherein the substance is at least one member selected from the group consisting of (a) a material incorporated in the dose in a manner that when the oral medication is properly administered is by itself or when combined with a reagent visible in normal light or when exposed to a special frequency light source, (b) a material that is by itself not readily visible in the excretion in normal light but by itself or when combined with a reagent is readily visible when exposed to a special frequency light source, (c) a material that is by itself not readily visible in the excretion in normal light but when combined with a reagent is readily visible in normal light and (d) a material that is by itself not readily visible in the excretion in normal light but by itself or when combined with a reagent is readily detected by a non-visual detection method.

28. The oral medication according to claim 27, wherein the substance is a micro radio tag or possesses a low level of radiation.

29. A method of promoting compliance with a risk evaluation and mitigation strategy comprising administering the dosage form of claim 27 and detecting the presence of the substance.

30. A dosage form comprising a dose of at least one bioactive agent and a substance that can be readily confirmed by medical personnel to determine if the patient is compliant with taking the dosage form, wherein the substance is at least one member selected from the group consisting of (a) a material incorporated in the dose in a manner that when the dosage form is properly administered is by itself or when combined with a reagent visible in the buccal, vaginal, nasal, rectal, aural, ophthalmic or ostomic cavity of a patient or an excretion of a patient in normal light or when exposed to a special frequency light source, (b) a material that is by itself not readily visible in the buccal, vaginal, nasal, rectal, aural, ophthalmic or ostomic cavity of a patient or an excretion of a patient in normal light but by itself or when combined with a reagent is readily visible when exposed to a special frequency light source, (c) a material that is by itself not readily visible in the buccal, vaginal, nasal, rectal, aural, ophthalmic or ostomic cavity of a patient or an excretion of a patient in normal light but when combined with a reagent is readily visible in normal light and (d) a material that is by itself not readily visible in the buccal, vaginal, nasal, rectal, aural, ophthalmic or ostomic cavity of a patient or an excretion of a patient in normal light but by itself or when combined with a reagent is readily detected by a non-visual detection method.

31. A method of using a Closed Loop Diagnostic System in order discern the presence of an analyte directly or by reference to another bound entity, comprising administering to a patient a dose of at least one bioactive agent and administering a substance that can be readily confirmed by medical personnel to determine if the patient is compliant with taking the dosage form, wherein the substance is at least one member selected from the group consisting of (a) a material incorporated in the dose in a manner that when the dosage form is properly administered is by itself or when combined with a reagent visible in the buccal, vaginal, nasal, rectal, aural, ophthalmic or ostomic cavity of a patient or an excretion of a patient in normal light or when exposed to a special frequency light source, (b) a material that is by itself not readily visible in the buccal, vaginal, nasal, rectal, aural, ophthalmic or ostomic cavity of a patient or an excretion of a patient in normal light but by itself or when combined with a reagent is readily visible when exposed to a special frequency light source, (c) a material that is by itself not readily visible in the buccal, vaginal, nasal, rectal, aural, ophthalmic or ostomic cavity of a patient or an excretion of a patient in normal light but when combined with a reagent is readily visible in normal light and (d) a material that is by itself not readily visible in the buccal, vaginal, nasal, rectal, aural, ophthalmic or ostomic cavity of a patient or an excretion of a patient in normal light but by itself or when combined with a reagent is readily detected by a non-visual detection method, and detecting the presence of the substance.
